(19) United States
(12) Patent Application Publication
(10) Pub. No.: US 2011/0263016 A1
(43) Pub. Date: Oct. 27, 2011

(54) Expansion of Embryonic Stem Cells

(76) Inventors: Derrick E. Rancourt, Calgary (CA); Guoliang Meng, Calgary (CA); Shiying Lui, Calgary (CA); Xiangyun Li, Hebei (CN); Roman Krawetz, Calgary (CA); Jaymi Cormier, Calgary (CA)

(21) Appl. No.: 13/120,832

(22) PCT Filed: Sep. 25, 2009
(86) PCT No.: PCT/IB2009/007100

§ 371 Date: Jul. 18, 2011

Related U.S. Application Data
(60) Provisional application No. 61/100,160, filed on Sep. 25, 2008.

(51) Int. Cl. C12N 5/0735; C12N 5/077; C12N 5/078; C12N 5/071; C12N 5/079

(52) U.S. Cl. 435/366; 435/325; 435/377; 435/378

(57) ABSTRACT

The present invention addresses the problem of expanding human embryonic stem cell (hESC) and induced pluripotent stem cell (iPSC) populations to clinically relevant numbers while maintaining pluripotency. In particular, the use of macrolide antibiotics and inhibitors of Rho and Rho-associated kinases is described.

This application claims benefit of priority to U.S. Provisional Application Ser. No. 61/100,160, filed Sep. 25, 2008, the entire contents of which are hereby incorporated by reference.

### BACKGROUND OF THE INVENTION

1. Field of the Invention

The present invention relates generally to the fields of cell biology, developmental biology and immunology. More particularly, it concerns methods and compositions relating to the expansion of stem cells, including human embryonic stem cells.

2. Description of Related Art

Human embryonic stem cells (hES cells or hESCs) have the ability to self-renew, and differentiate into multilineage cell types from all three embryonic layers. These properties make hESCs attractive as a source for cell therapy, regenerative medicine, and basic scientific research.

In the transition of hESCs from the research lab to clinical application, robust culture systems that consistently control stem cell growth and differentiation are required. Stem cell-based processes must reproducibly generate large amounts of functional cells. Therefore, maintaining the stem cell microenvironment is key to this process. There are inherent problems associated with hESCs expanded in static culture. For example, hES cells are sensitive to trypsinization, which can lead to cell death or apoptosis. Further, the detrimental effects of hydrodynamic shear (generated in stirred suspension culture systems) must be balanced with the need for adequate oxygen transfer to the cells as well as the necessity to control cell aggregation. Achieving growth of hESCs in bioreactors is important because a bioreactor produces clinically relevant cell numbers on a large scale (e.g., on the order of 10⁶ cells/ml) and these systems can be easily adapted to meet GMP level standards.

Therefore, the development of improved methods for growth and expansion of hESCs is urgently needed.

### SUMMARY OF THE INVENTION

Thus, in accordance with the present invention, there is provided a method for expanding human embryonic stem cells (hESCs) and induced pluripotent stem cells (iPSCs) in culture comprising (a) providing a starting culture comprising hESCs or iPSCs; (b) incubating said starting culture with a macrolide antibiotic and a Rho and Rho family kinase inhibitor for a period of time and under conditions suitable to permit expansion of said hESCs or iPSCs. The macrolide antibiotic may be selected from the group consisting of sirolimus (Rapamycin), tacrolimus, cyclosporine, everolimus, ascomycin, erythromycin, azithromycin, clarithromycin, clindamycin, lincomycin, dirithromycin, josamycin, spiramycin, diacetyl-midecamycin, tylosin, roxithromycin, ABT-773, telithromycin, leucomycins and lincosamide. The starting culture may be comprised in a bioreactor or a static vessel. The bioreactor may be a micro-bioreactor, or a commercial bioreactor. The bioreactor may have a volume of between 100 μl and 1000 L. Step (a) may comprise preparing said starting culture by inoculating said bioreactor with hESCs or iPSCs from a static culture.

The macrolide antibiotic may be present at about 0.1 nM to 100 nM. The period of time may comprise 2, 3, 4, 5, 6, 7, 8, 9, 10, 11, 12, 13, 14, 15, 16, 17, 18, 19, 20, 21, 22, 23, 24, 25, 30, 40 or 50 days. Incubating may comprise of periodic treatment with dissociation agents to reduce aggregate size. Dissociation may be achieved through treatment with compounds such as collagenase, trypsin, dispase, versene, or triple or Accutase. The Rho and Rho family kinase inhibitor may be a ROCK inhibitor or C-3 toxin. The Rho and Rho family kinase inhibitor may be removed at about 1 day following initiation of incubation. The starting culture may comprise about 10⁴ to 10⁷ cells. Expansion may comprise 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 60-fold, 70-fold, 80-fold, 90-fold, 100-fold, 125-fold, 150-fold, 200-fold, 400-fold, 500-fold or 1000-fold expansion.

The method may further comprise inducing said hESCs or iPSCs to differentiate, such as into immune cells, neuronal cells, cardiovascular cells, muscular cells, skeletal cells, islet cells, bone cells, or cartilage cells. The starting culture may comprise mTeSR medium or animal-free medium or xeno-free medium.

In another embodiment, there is provided a method for expanding embryonic stem cells (ESCs) or induced pluripotent stem cells (iPSCs) in culture comprising (a) providing a bioreactor comprising a culture media, hESCs or iPSCs and a macrolide antibiotic; (b) incubating said bioreactor for a period of time and under conditions suitable to permit expansion of said hESCs or iPSCs by about 10- to 1000-fold.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### BRIEF DESCRIPTION OF THE DRAWINGS

The following drawings faun part of the present specification and are included to further demonstrate certain aspects of the present invention. The invention may be better understood by reference to one or more of these drawings in combination with the detailed description of specific embodiments presented herein.

FIGS. 1A-E. (FIG. 1A) Human embryonic stem cells were expanded in suspension bioreactors without passaging but with repeated treatment with collagenase every 5 days (dotted lines). These cultures were treated with ROCK inhibitor from day 0 until day 4 (arrow indicates removal of ROCK inhibitor). (FIG. 1B) Bright-field photomicrographs of hESCs on days 1, 5, 15 and 25 of expansion in the bioreactors. (FIG. 1C) Immunofluorescent detection of Toto3 (nuclear marker), Oct-4, SSEA-4, TRA-1-60 and TRA-1-81 in hESC bioreactor cultures on days 5 and 20 of expansion (protein markers are shown in green and cell nuclei in blue). The corresponding FACS data for each marker is also shown. (FIG. 1D) Karyotype of hESC bioreactor cultures on day 20 of expansion. (FIG. 1E) Teratomas generated from day 20 hESC bioreactor cultures; sections stained with H&E.

FIGS. 2A-C. (FIG. 2A) Human embryonic stem cells derived from static Matrigel™ or HFF cultures. The bioreactor cultures were passaged every 6 days by dissociating the aggregates into single cells and splitting each culture 1:5. (FIG. 2B) A comparison of the growth kinetics of the hESC passaged and non-passaged bioreactor cultures over the first 5 days. Passaged cultures were treated with ROCK inhibitor for 24 hours only (*) whereas non-passaged cultures were treated with ROCK until day 4 (**). (FIG. 2C) A comparison of murine and human stem cell growth kinetics in bioreactor culture systems (Cormier et al., 2006).

FIGS. 3A-C. Characterization of hESC aggregates from bioreactor cultures that were repeatedly passaged. (FIG. 3A) Immunofluorescent analysis of pluripotency marker expression (Oct-4, Nanog, TRA-1-60, TRA-1-81, SSEA-4) in day 21 hESC aggregates from bioreactor cultures. Representative bioreactor aggregates from both Matrigel™ and HFF-derived hESC cultures display similar localization of each marker tested, with uniform distribution of staining with the aggregates (inset). (FIG. 3B) hESCs maintained on Matrigel™ or HFFs before bioreactor seeding display no difference in expression of pluripotency markers on day 6 of culture as analyzed by FACS. FACS analysis shows that markers on day 21 remain at high levels similar to day 6. (FIG. 3C) hESCs retained a normal karyotype (46, XX) as well as the ability to generate teratomas containing tissues derived from all three germ layers in vivo after 21 days of bioreactor culture.

FIGS. 4A-C. Animal/Xeno-free culture of human embryonic stem cells in suspension bioreactor. H9 hESCs were cultured using TF1 animal/xeno-free medium for 96 hours in a stirred suspension bioreactor using 0.1 nM Rapamycin constantly, and 10 μM Y-27632 for the first 24 hours. (FIG. 4A) Viable cell density over the first 96 hours. Morphology of the cells at 24 hours (FIG. 4B) and 96 hours (FIG. 4C) post-inoculation. Scale bar represents 25

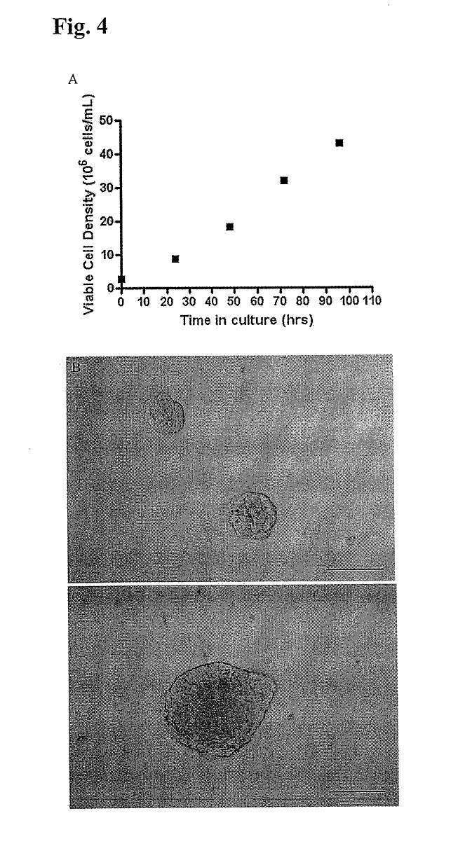

FIGS. 5A-B. Human induced pluripotent stem cells (iPSCs) expanded in suspension bioreactor in TF1 media. Animal/Xeno-free culture of iPSCs in suspension bioreactor. iPSCs were cultured using TF1 animal/xeno-free medium for 48 hours in a stirred suspension bioreactor using 0.1 nM Rapamycin constantly, and 10 μM Y-27632 for the first 24 hours. Viable cell density over the first 48 hours (FIG. 5A). Morphology of the cells at 48 hours (FIG. 5B) post-inoculation.

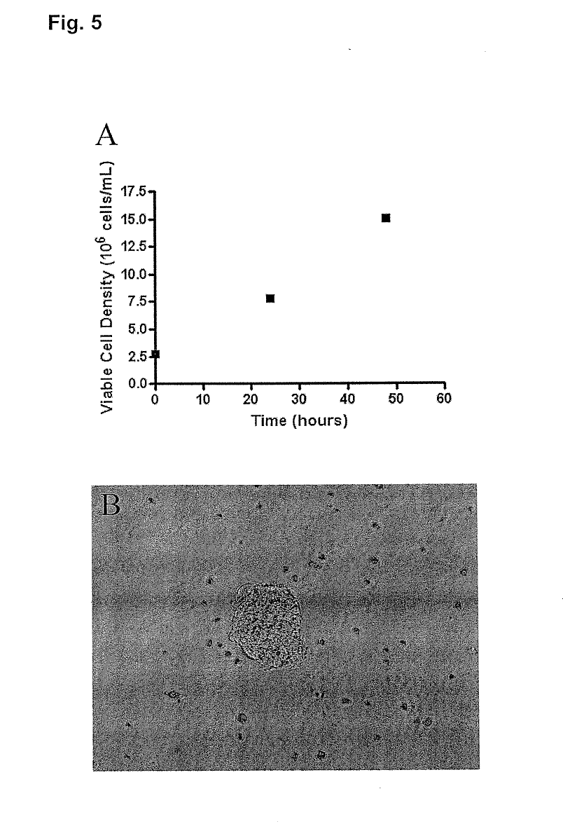

### DESCRIPTION OF ILLUSTRATIVE EMBODIMENTS

More than a decade ago, Thomson et al. (1998) derived the first human embryonic stem cell (hESC) line. Since this initial discovery, a number of studies have sought to evolve hESC technology to the point were hESCs can be utilized for transplantation. The development of successful clinical therapies using hESCs will depend on the establishment of technologies for the large-scale expansion and controlled differentiation of the cells (King and Miller, 2007). Currently, hESCs are cultured in static tissue culture flasks, which are disadvantageous as they result in culture heterogeneity, low reproducibility due to the lack of control of the culture conditions, and low cell yield. Suspension bioreactors are an effective alternative to static tissue culture flasks as these vessels enable the generation of clinically relevant cell numbers in one culture system, eliminate culture-to-culture variation, and permit control and monitoring of culture conditions including oxygen, pH and temperature. In addition, the stirred environment results in homogeneous culture conditions and can facilitate cell seeding onto scaffolds, enabling the creation of tissue constructs with greater efficacy. Importantly, the use of suspension bioreactors also enables the expansion of hESCs in the absence of feeder layers or matrices, which will facilitate the adaptation of GMP standards to the development of cell therapies.

In the current study, the inventors successfully developed bioreactor protocols for hESCs and achieved a 67-fold expansion over 20 days. They further showed that the hESCs could be regularly passaged in the bioreactors enabling long term expansion of the cells. Using immunofluorescent staining, flow cytometry and teratoma formation assays, the inventors demonstrated that the hESC bioreactor cultures retained high levels of pluripotency and also had a normal karyotype after regular passaging. These expanded hESCs appear to cells retain their ability to differentiate, which is an essential property when expanding hESC populations. In the absence of the antibiotic, the hESCs die within two days. Thus, the inventors propose the use of macrolide antibiotics in conjunction with other culturing parameters (Rho and Rho kinase inhibitors, bioreactors) to achieve significant expansion of hESCs or induced pluripotent stem cells (iPSCs), as well as their subsequent use in culture-induced differentiation. These, and other aspects of the invention, are set out in detail below.

### I. STEM CELLS AND CELL LINES

A. Stem Cells

Stem cells are primal cells found in all multi-cellular organisms that retain the ability to self-renew themselves through mitotic cell division and can differentiate into a diverse range of specialized cell types. The three broad categories of mammalian stem cells are embryonic stem cells (derived from blastocysts), adult stem cells (found in adult tissues), and cord blood stem cells (found in the umbilical cord). In a developing embryo, stem cells can differentiate into all of the specialized embryonic tissues. In adult organisms, stem cells and progenitor cells act as a repair system for the body, replenishing specialized cells.

As stem cells can be readily grown and transformed into specialized cells with characteristics consistent with cells of various tissues such as muscles or nerves through cell culture, their use in medical therapies has been proposed. In particular, embryonic cell lines, autologous embryonic stem cells generated through therapeutic cloning, and highly plastic adult stem cells from the umbilical cord blood or bone marrow are touted as promising candidates.

i. Embryonic Stem Cells (ESCs)

ESCs are cells derived from the epiblast tissue of the inner cell mass (ICM) of a blastocyst. A blastocyst is an early stage embryo—approximately 4 to 5 days-old in humans and consisting of 50-150 cells. ESCs are pluripotent, and give rise during development to all derivatives of the three primary gemi layers: ectoderm, endoderm and mesoderm. In other words, they can develop into each of the more than 200 cell types of the adult body when given sufficient and necessary stimulation for a specific cell type. They do not contribute to the extra-embryonic membranes or the placenta.

A human embryonic stem cell is defined by the presence of several transcription factors and cell surface proteins. The transcription factors Oct-4, Nanog, and Sox2 form the core regulatory network which ensures the suppression of genes that lead to differentiation and the maintenance of pluripotency. The cell surface proteins most commonly used to identify hESCs are the glycolipids SSEA3 and SSEA4 and the keratan sulfate antigens Tra-1-60 and Tra-1-81. The molecular definition of a stem cell includes many more proteins and continues to be a topic of research.

ii. Adult Stem Cells

Adult stem cells are undifferentiated cells found throughout the body that divide to replenish dying cells and regenerate damaged tissues. Also known as somatic stem cells, they can be found in children, as well as adults. A great deal of adult stem cell research has focused on clarifying their capacity to divide or self-renew indefinitely and their differentiation potential. Adult stem cells, like embryonic stem cells, have pluripotent potential and can differentiate into cells derived from all three germ layers. In mice, pluripotent stem cells can be directly generated from adult fibroblast cultures.

While embryonic stem cell potential remains untested, adult stem cell treatments have been used for many years to successfully treat leukemia and related bone/blood cancers through bone marrow transplants. The use of adult stem cells in research and therapy is not as controversial as embryonic stem cells, because the production of adult stem cells does not require the destruction of an embryo.

iii. Endothelial Progenitor Cells

Endothelial progenitor cells (EPCs) are bone marrow-derived cells that circulate in the blood and have the ability to differentiate into endothelial cells, the cells that make up the lining of blood vessels. The process by which blood vessels are born de novo from EPCs is known as vasculogenesis. Most of vasculogenesis occurs in utero during embryologic development. EPCs found in adults are thus related to angioblasts, which are the stem cells that form blood vessels during embryogenesis. EPCs are thought to participate in pathologic angiogenesis such as that found in retinopathy and tumor growth. While angioblasts have been known to exist for many years, adult EPCs were only characterized in the 1990's after Asahara and colleagues published that a purified population of CD34-expressing cells isolated from the blood of adult mice could differentiate into endothelial cells in vitro. As EPCs are originally derived from the bone marrow, it is thought that various cytokines, growth factors, and hormones cause them to be mobilized from the bone marrow and into the peripheral circulation where they ultimately are recruited to regions of angiogenesis.

In animal models of myocardial infarction, the injection of ex vivo expanded EPCs or stem and progenitor cells significantly improved blood flow and cardiac function and reduced left ventricular scarring. Similarly, infusion of ex vivo expanded EPCs deriving from peripheral blood mononuclear cells in nude mice or rats improved the neovascularization in hind limb ischemia models. Correspondingly, initial pilot trials indicate that bone marrow-derived or circulating blood-derived progenitor cells are useful for therapeutically improving blood supply of ischemic tissue. In addition, transplantation of ex vivo expanded EPCs significantly improved coronary flow reserve and left ventricular function in patients with acute myocardial infarction.

Of the three cell markers (CD133, CD34, and the vascular endothelial growth factor receptor 2) that characterize the early functional EPCs in adult bone marrow, EPCs lose CD133/CD34 and start to express CD31, vascular endothelial cadherin, and von Willebrand factor when migrating to the circulation. Various isolation procedures of EPCs from different sources by using adherence culture or affinity magnetic microbeads have been described (e.g., WO/2005/078073; Asahara et al., 1997; Asahara et al., 1999; Hristov et al., 2003; Shaw et al., 2004; Werner et al., 2005).

iv. Induced Pluripotent Stem Cells

Induced pluripotent stem cells, commonly abbreviated as iPS cells or iPSCs, are a type of pluripotent stem cell artificially derived from a non-pluripotent cell, typically an adult somatic cell, by inducing a forced expression of certain genes. iPSCs are believed to be identical to natural pluripotent stem cells, such as embryonic stem cells in many respects, such as the expression of certain stem cell genes and proteins, chromatin methylation patterns, doubling time, embryoid body formation, teratoma formation, viable chimera formation, and potency and differentiability, but the full extent of their relation to natural pluripotent stem cells is still being assessed.

iPSCs were first produced in 2006 from mouse cells and in 2007 from human cells. This has been cited as an important advancement in stem cell research, as it may allow researchers to obtain pluripotent stem cells, which are important in research and potentially have therapeutic uses, without the controversial use of embryos. iPSCs are typically derived by transfection of certain stem cell-associated genes into non-pluripotent cells, such as adult fibroblasts. Transfection is typically achieved through viral vectors, such as retroviruses. Transfected genes include the master transcriptional regulators Oct-3/4 (Pouf51) and Sox2, although it is suggested that other genes enhance the efficiency of induction. After 3-4 weeks, small numbers of transfected cells begin to become morphologically and biochemically similar to pluripotent stem cells, and are typically isolated through morphological selection, doubling time, or through a reporter gene and antibiotic selection. Eventually, four key pluripotency genes essential for the production of pluripotent stem cells were isolated: Oct-3/4, Sox2, c-Myc, and Klf4. In a later study, Yamanaka reported that one can create iPSCs even without c-Myc. The process takes longer and is not as efficient, but the resulting chimeras did not develop cancer.

In November 2007, iPSCs were created from adult human cells; two independent research teams' studies were released—one in Science by James Thomson and colleagues at University of Wisconsin-Madison and another in Cell by Shinya Yamanaka and colleagues at Kyoto University, Japan. With the same principle used earlier in mouse models, Yamanaka had successfully transformed human fibroblasts into pluripotent stem cells using the same four pivotal genes: Oct3/4, Sox2, Klf4, and c-Myc with a retroviral system. Thomson and colleagues used Oct4, Sox2, Nanog, and a different gene LIN28 using a lentiviral system.

Oct-3/4 and certain members of the Sox gene family (Sox1, Sox2, Sox3, and Sox15) have been identified as crucial transcriptional regulators involved in the induction process whose absence makes induction impossible. Additional genes, however, including certain members of the Klf family (Klf1, Klf2, Klf4, and Klf5), the Myc family (c-Myc, 1-Myc, and n-Myc), Nanog, and LIN28, have been identified to increase the induction efficiency.
Oct-3/4 (Pou5fl): Oct-3/4 is one of the family of octamer (“Oct”) transcription factors, and plays a crucial role in maintaining pluripotency. The absence of Oct-3/4 in Oct-3/4⁺ cells, such as blastomeres and embryonic stem cells, leads to spontaneous trophoblast differentiation, and presence of Oct-3/4 thus gives rise to the pluripotency and differentiation potential of embryonic stem cells. Various other genes in the “Oct” family, including Oct-3/4's close relatives, Oct-1 and Oct-6, fail to elicit induction, thus demonstrating the exclusiveness of Oct-3/4 to the induction process.
Sox family: The Sox family of genes is associated with maintaining pluripotency similar to Oct-3/4, although it is associated with multipotent and unipotent stem cells in contrast with Oct-3/4, which is exclusively expressed in pluripotent stem cells. While Sox2 was the initial gene used for induction, other genes in the Sox family have been found to work as well in the induction process. Sox1 yields iPSCs with a similar efficiency as Sox2, and genes Sox3, Sox15, and Sox18 also generate iPSCs, although with decreased efficiency.
Klf family: Klf4 of the Klf family of genes was initially identified as a factor for the generation of mouse iPSCs and was demonstrated as a factor for generation of human iPSCs. However, Klf4 was found unnecessary for generation of human iPSCs and in fact failed to generate human iPSCs. Klf2 and Klf4 were found to be factors capable of generating iPSCs, and related genes Klf1 and Klf5 did as well, although with reduced efficiency.
Myc family: The Myc family of genes contains proto-oncogenes implicated in cancer. Early reports demonstrated that c-Myc is a factor implicated in the generation of mouse iPSCs and human iPSCs. However, later studies showed that c-myc was unnecessary for generation of human iPSCs. Usage of the myc family of genes in induction of iPSCs is troubling for the eventuality of iPSCs as clinical therapies, as 25% of mice transplanted with c-myc-induced iPSCs developed lethal teratomas. n-Myc and 1-Myc have been identified to induce in the stead of c-Myc with similar efficiency.
Nanog: In embryonic stem cells, Nanog, along with Oct-3/4 and Sox2, is necessary in promoting pluripotency. Therefore, it was surprising when Nanog was reported to be unnecessary for induction.
LIN28: LIN28 is an mRNA binding protein expressed in embryonic stem cells and embryonic carcinoma cells associated with differentiation and proliferation, and has been shown to be a factor in iPSC generation, although it is unnecessary.
   Generated iPSCs are remarkably similar to naturally-isolated pluripotent stem cells (such as mouse and human embryonic stem cells, mESCs and hESCs, respectively) in the following respects, thus confirming the identity, authenticity, and pluripotency of iPSCs to naturally-isolated pluripotent stem cells. iPSCs expressed cell surface antigenic markers expressed on ESCs. Human iPSCs expressed the markers specific to hESC, including SSEA-3, SSEA-4, TRA-1-60, TRA-1-81, TRA-2-49/6E, and Nanog. Mouse iPSCs expressed SSEA-1 but not SSEA-3 nor SSEA-4, similarly to mESCs. iPSCs are capable of differentiation in a fashion similar to ESCs into fully differentiated tissues, such as neural, cardiac, and teratoma tissues, as well as blastocysts.

B. Obtaining Primary Stem Cells

Methods of separating primary stem cell populations are well known in the art and may be applied to the cell populations of the present invention. Embryonic stem cells, as well as stem cells for neuronal, endothelial, cardiac and other cell types express various surface antigens and, in fact, are often classified on this basis. These antigens provide targets by which the skilled artisan can identify and separate stems cells from non-stem cells, and even separate various stem cell populations from each other.

TABLE 1
MARKERS COMMONLY USED TO IDENTIFY STEM CELLS AND TO
CHARACERTIZE DIFFERENTIATED CELL TYPES

| | | |
|---|---|---|
| Marker Name | Cell Type | Significance |

Blood Vessel

| | | |
|---|---|---|
| Fetal liver kinase- | Endothelial | Cell-surface receptor protein that identifies |
| 1 (Flk1) | | endothelial cell progenitor; marker of cell- |
| | | cell contacts |
| Smooth muscle | Smooth muscle | Identifies smooth muscle cells in the wall of |
| cell-specific | | blood vessels |
| myosin heavy | | |
| chain | | |
| Vascular | Smooth muscle | Identifies smooth muscle cells in the wall of |
| endothelial cell | | blood vessels |
| cadherin | | |

Bone

| | | |
|---|---|---|
| Bone-specific | Osteoblast | Enzyme expressed in osteoblast; activity |
| alkaline | | indicates bone formation |
| phosphatase | | |
| (BAP) | | |
| Hydroxyapatite | Osteoblast | Mineralized bone matrix that provides |
| | | structural integrity; marker of bone |
| | | formation |
| Osteocalcin (OC) | Osteoblast | Mineral-binding protein uniquely |
| | | synthesized by osteoblast; marker of bone |
| | | formation |

Bone Marrow and Blood

| | | |
|---|---|---|
| Bone | Mesenchymal stem | Important for the differentiation of |
| morphogenetic | and progenitor cells | committed mesenchymal cell types from |
| protein receptor | | mesenchymal stem and progenitor cells; |
| (BMPR) | | BMPR identifies early mesenchymal |
| | | lineages (stem and progenitor cells) |
| CD4 and CD8 | White blood cell | Cell-surface protein markers specific for |
| | (WBC) | mature T lymphocyte (WBC subtype) |
| CD34 | Hematopoietic stem | Cell-surface protein on bone marrow cell, |
| | cell (HSC), satellite, | indicative of a HSC and endothelial |
| | endothelial | progenitor; CD34 also identifies muscle |
| | progenitor | satellite, a muscle stem cell |
| CD34⁺Sca1⁺ Lin⁻ | Mesencyhmal stem | Identifies MSCs, which can differentiate into |
| profile | cell (MSC) | adipocyte, osteocyte, chondrocyte, and |
| | | myocyte |
| CD38 | Absent on HSC | Cell-surface molecule that identifies WBC |
| | Present on WBC | lineages. Selection of CD34⁺/CD38⁻ cells |
| | lineages | allows for purification of HSC populations |
| CD44 | Mesenchymal | A type of cell-adhesion molecule used to |
| | | identify specific types of mesenchymal cells |
| c-Kit | HSC, MSC | Cell-surface receptor on bone marrow cell |
| | | (BMC) types that identifies HSCs and |
| | | MSCs; binding by fetal calf serum (FCS) |
| | | enhances proliferation of ESCs, HSCs, |
| | | MSCs, and hematopoietic progenitor cells |
| Colony-forming | HSC, MSC | CFU assay detects the ability of a single stem |
| unit (CFU) | progenitor | cell or progenitor cell to give rise to one or |
| | | more cell lineages, such as red blood cell |
| | | (RBC) and/or white blood cell (WBC) |
| | | lineages |
| Fibroblast colony- | Bone marrow | An individual bone marrow cell that has |
| forming unit | fibroblast | given rise to a colony of multipotent |
| (CFU-F) | | fibroblastic cells; such identified cells are |
| | | precursors of differentiated mesenchymal |
| | | lineages |
| Hoechst dye | Absent on HSC | Fluorescent dye that binds DNA; HSC |
| | | extrudes the dye and stains lightly compared |
| | | with other cell types |
| Leukocyte | WBC | Cell-surface protein on WBC progenitor |
| common antigen | | |
| (CD45) | | |
| Lineage surface | HSC, MSC | Thirteen to 14 different cell-surface proteins |
| antigen (Lin) | Differentiated RBC | that are markers of mature blood cell |
| | and WBC lineages | lineages; detection of Lin-negative cells |
| | | assists in the purification of HSC and |
| | | hematopoietic progenitor populations |
| Mac-1 | WBC | Cell-surface protein specific for mature |
| | | granulocyte and macrophage (WBC |
| | | subtypes) |
| Muc-18 (CD146) | Bone marrow | Cell-surface protein (immunoglobulin super |
| | fibroblasts, | family) found on bone marrow fibroblasts, |
| | endothelial | which may be important in hematopoiesis; a |
| | | subpopulation of Muc-18+ cells are |
| | | mesenchymal precursors |
| Stem cell antigen | HSC, MSC | Cell-surface protein on BMCs, indicative of |
| (Sca-1) | | HSC and MSC Bone Marrow and Blood |
| | | cont. |
| Stro-1 antigen | Stromal | Cell-surface glycoprotein on subsets of bone |
| | (mesenchymal) | marrow stromal (mesenchymal) cells; |
| | precursor cells, | selection of Stro-1+ cells assists in isolating |
| | hematopoietic cells | mesenchymal precursor cells, which are |
| | | multipotent cells that give rise to adipocytes, |
| | | osteocytes, smooth myocytes, fibroblasts, |
| | | chondrocytes, and blood cells |
| Thy-1 | HSC, MSC | Cell-surface protein; negative or low |
| | | detection is suggestive of HSC |

Cartilage

| | | |
|---|---|---|
| Collagen types II | Chondrocyte | Structural proteins produced specifically by |
| and IV | | chondrocyte |
| Keratin | Keratinocyte | Principal protein of skin; identifies |
| | | differentiated keratinocyte |
| Sulfated | Chondrocyte | Molecule found in connective tissues; |
| proteoglycan | | synthesized by chondrocyte |

Fat

| | | |
|---|---|---|
| Adipocyte lipid- | Adipocyte | Lipid-binding protein located specifically in |
| binding protein | | adipocyte |
| (ALBP) | | |
| Fatty acid | Adipocyte | Transport molecule located specifically in |
| transporter (FAT) | | adipocyte |
| Adipocyte lipid- | Adipocyte | Lipid-binding protein located specifically in |
| binding protein | | adipocyte |
| (ALBP) | | |

General

| | | |
|---|---|---|
| Y chromosome | Male cells | Male-specific chromosome used in labeling |
| | | and detecting donor cells in female transplant |
| | | recipients |
| Karyotype | Most cell types | Analysis of chromosome structure and |
| | | number in a cell |

Liver

| | | |
|---|---|---|
| Albumin | Hepatocyte | Principal protein produced by the liver; |
| | | indicates functioning of maturing and fully |
| | | differentiated hepatocytes |
| B-1 integrin | Hepatocyte | Cell-adhesion molecule important in cell-cell |
| | | interactions; marker expressed during |
| | | development of liver |

Nervous System

| | | |
|---|---|---|
| CD133 | Neural stem cell, | Cell-surface protein that identifies neural |
| | HSC | stem cells, which give rise to neurons and |
| | | glial cells |
| Glial fibrillary | Astrocyte | Protein specifically produced by astrocyte |
| acidic protein | | |
| (GFAP) | | |
| Microtubule- | Neuron | Dendrite-specific MAP; protein found |
| associated protein- | | specifically in dendritic branching of neuron |
| 2 (MAP-2) | | |
| Myelin basic | Oligodendrocyte | Protein produced by mature |
| protein (MPB) | | oligodendrocytes; located in the myelin |
| | | sheath surrounding neuronal structures |
| Nestin | Neural progenitor | Intermediate filament structural protein |
| | | expressed in primitive neural tissue |
| Neural tubulin | Neuron | Important structural protein for neuron; |
| | | identifies differentiated neuron |
| Neurofilament | Neuron | Important structural protein for neuron; |
| (NF) | | identifies differentiated neuron |
| Neurosphere | Embryoid body | Cluster of primitive neural cells in culture of |
| | (EB), ESC | differentiating ESCs; indicates presence of |
| | | early neurons and glia |
| Noggin | Neuron | A neuron-specific gene expressed during the |
| | | development of neurons |
| O4 | Oligodendrocyte | Cell-surface marker on immature, |
| | | developing oligodendrocyte |
| O1 | Oligodendrocyte | Cell-surface marker that characterizes mature |
| | | oligodendrocyte |
| Synaptophysin | Neuron | Neuronal protein located in synapses; |
| | | indicates connections between neurons |
| Tau | Neuron | Type of MAP; helps maintain structure of |
| | | the axon |

Pancreas

| | | |
|---|---|---|
| Cytokeratin 19 | Pancreatic | CK19 identifies specific pancreatic epithelial |
| (CK19) | epithelium | cells that are progenitors for islet cells and |
| | | ductal cells |
| Glucagon | Pancreatic islet | Expressed by alpha-islet cell of pancreas |
| Insulin | Pancreatic islet | Expressed by beta-islet cell of pancreas |
| Insulin-promoting | Pancreatic islet | Transcription factor expressed by beta-islet |
| factor-1 (PDX-1) | | cell of pancreas |
| Nestin | Pancreatic progenitor | Structural filament protein indicative of |
| | | progenitor cell lines including pancreatic |
| Pancreatic | Pancreatic islet | Expressed by gamma-islet cell of pancreas |
| polypeptide | | |
| Somatostatin | Pancreatic islet | Expressed by delta-islet cell of pancreas |

Pluripotent Stem Cells

| | | |
|---|---|---|
| Alkaline | Embryonic stem cell | Elevated expression of this enzyme is |
| phosphatase | (ESC), embryonal | associated with undifferentiated pluripotent |
| | carcinoma (EC) | stem cells (PSCs) |
| Alpha-fetoprotein | Endoderm | Protein expressed during development of |
| (AFP) | | primitive endoderm; reflects endodermal |
| | | differentiation of PSCs |
| Bone | Mesoderm | Growth and differentiation factor expressed |
| morphogenetic | | during early mesoderm formation and |
| protein-4 | | differentiation |
| Brachyury | Mesoderm | Transcription factor important in the earliest |
| | | phases of mesoderm formation and |
| | | differentiation; used as the earliest indicator |
| | | of mesoderm formation |
| Cluster | ESC, EC | Surface receptor molecule found specifically |
| designation 30 | | on PSCs |
| (CD30) | | |
| Cripto (TDGF-1) | ESC, cardiomyocyte | Gene for growth factor expressed by ESCs, |
| | | primitive ectoderm, and developing |
| | | cardiomyocyte |
| GATA-4 gene | Endoderm | Expression increases as ESC differentiates |
| | | into endoderm |
| GCTM-2 | ESC, EC | Antibody to a specific extracellular-matrix |
| | | molecule that is synthesized by |
| | | undifferentiated PSCs |
| Genesis | ESC, EC | Transcription factor uniquely expressed by |
| | | ESCs either in or during the undifferentiated |
| | | state of PSCs |
| Germ cell nuclear | ESC, EC | Transcription factor expressed by PSCs |
| factor | | |
| Hepatocyte | Endoderm | Transcription factor expressed early in |
| nuclear factor-4 | | endoderm formation |
| (HNF-4) | | |
| Nestin | Ectoderm, neural and | Intermediate filaments within cells; |
| | pancreatic progenitor | characteristic of primitive neuroectoderm |
| | | formation |
| Neuronal cell- | Ectoderm | Cell-surface molecule that promotes cell-cell |
| adhesion molecule | | interaction; indicates primitive |
| (N-CAM) | | neuroectoderm formation |
| Pax6 | Ectoderm | Transcription factor expressed as ESC |
| | | differentiates into neuroepithelium |
| Stage-specific | ESC, EC | Glycoprotein specifically expressed in early |
| embryonic | | embryonic development and by |
| antigen-3 (SSEA- | | undifferentiated PSCs |
| 3) | | |
| Stage-specific | ESC, EC | Glycoprotein specifically expressed in early |
| embryonic | | embryonic development and by |
| antigen-4 (SSEA- | | undifferentiated PSCs |
| 4) | | |
| Stem cell factor | ESC, EC, HSC, | Membrane protein that enhances |
| (SCF or c-Kit | MSC | proliferation of ESCs and EC cells, |
| ligand) | | hematopoietic stem cell (HSCs), and |
| | | mesenchymal stem cells (MSCs); binds the |
| | | receptor c-Kit |
| Telomerase | ESC, EC | An enzyme uniquely associated with |
| | | immortal cell lines; useful for identifying |
| | | undifferentiated PSCs |
| TRA-1-60 | ESC, EC | Antibody to a specific extracellular matrix |
| | | molecule is synthesized by undifferentiated |
| | | PSCs |
| TRA-1-81 | ESC, EC | Antibody to a specific extracellular matrix |
| | | molecule normally synthesized by |
| | | undifferentiated PSCs |
| Vimentin | Ectoderm, neural and | Intermediate filaments within cells; |
| | pancreatic progenitor | characteristic of primitive neuroectoderm |
| | | formation |

Skeletal Muscle/Cardiac/Smooth Muscle

| | | |
|---|---|---|
| MyoD and Pax7 | Myoblast, myocyte | Transcription factors that direct |
| | | differentiation of myoblasts into mature |
| | | myocytes |
| Myogenin and | Skeletal myocyte | Secondary transcription factors required for |
| MR4 | | differentiation of myoblasts from muscle |
| | | stem cells |
| Myosin heavy | Cardiomyocyte | A component of structural and contractile |
| chain | | protein found in cardiomyocyte |
| Myosin light chain | Skeletal myocyte | A component of structural and contractile |
| | | protein found in skeletal myocyte |

The following description sets forth exemplary methods for separation of stem cells based upon the surface expression of various markers.

i. Fluorescence Activated Cell Sorting (FACS)

FACS facilitates the quantitation and/or separation of subpopulations of cells based upon surface markers. Cells to be sorted are first tagged with a fluorescently labeled antibody or other marker specific ligand. Generally, labeled antibodies and ligands are specific for the expression of a phenotype specific cell surface molecule. The labeled cells are then passed through a laser beam and the fluorescence intensity of each cell determined. The sorter distributes the positive and negative cells into label-plus and label-minus wells at a flow rate of approximately 3000 cells per second.

The use of multiple fluorescent tags exciting at different wavelengths allows for sorting based upon multiple or alternate criteria. Among the fluorescent labels contemplated for use as conjugates include Alexa 350, Alexa 430, AMCA, BODIPY 630/650, BODIPY 650/665, BODIPY-FL, BODIPY—R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red. Thus, for example, a single PBMC sample may be analyzed with alternatively labeled anti-Ig antibody, anti-CD3 antibody, anti-CD8 antibody and anti-CD4 antibody to screen for the presence of B cells and T cells within the sample, as well as distinguishing specific T cell subsets.

FACS analysis and cell sorting is carried out on a flow cytometer. A flow cytometer generally consists of a light source, normally a laser, collection optics, electronics and a computer to translate signals to data. Scattered and emitted fluorescent light is collected by two lenses (one positioned in front of the light source and one set at right angles) and by a series of optics, beam splitters and filters, which allow for specific bands of fluorescence to be measured.

Flow cytometer apparatus permit quantitative multiparameter analysis of cellular properties at rates of several thousand cells per second. These instruments provide the ability to differentiate among cell types. Data are often displayed in one-dimensional (histogram) or two-dimensional (contour plot, scatter plot) frequency distributions of measured variables. The partitioning of multiparameter data files involves consecutive use of the interactive one- or two-dimensional graphics programs.

Quantitative analysis of multiparameter flow cytometric data for rapid cell detection consists of two stages: cell class characterization and sample processing. In general, the process of cell class characterization partitions the cell feature into cells of interest and not of interest. Then, in sample processing, each cell is classified in one of the two categories according to the region in which it falls. Analysis of the class of cells is very important, as high detection performance may be expected only if an appropriate characteristic of the cells is obtained.

Not only is cell analysis performed by flow cytometry, but so too is sorting of cells. In U.S. Pat. No. 3,826,364, an apparatus is disclosed which physically separates particles, such as functionally different cell types. In this machine, a laser provides illumination which is focused on the stream of particles by a suitable lens or lens system so that there is highly localized scatter from the particles therein. In addition, high intensity source illumination is directed onto the stream of particles for the excitation of fluorescent particles in the stream. Certain particles in the stream may be selectively charged and then separated by deflecting them into designated receptacles. A classic form of this separation is via fluorescent tagged antibodies, which are used to mark one or more cell types for separation.

Additional and alternate methods for performing flow cytometry and fluorescent antibody cell sorting are described in U.S. Pat. Nos. 4,284,412; 4,989,977; 4,498,766; 5,478,722; 4,857,451; 4,774,189; 4,767,206; 4,714,682; 5,160,974; and 4,661,913, herein expressly incorporated by reference.

ii. Micro-Bead Separation

Cells in suspension may be separated to very high purity according to their surface antigens using micro-bead technologies. The basic concept in micro-bead separations is to selectively bind the biomaterial of interest (e.g., a specific cell, protein, or DNA sequence) to a particle and then separate it from its surrounding matrix. Micro-bead separation involves contacting a cell suspension with a slurry of microbeads labeled with a cell surface specific antibody or ligand. Cells labeled with the micro-beads are then separated using an affinity capture method specific for some property of the beads. This format facilitates both positive and negative selection.

Magnetic beads are uniform, superparamagnetic beads generally coated with an affinity tag such as recombinant streptavidin that will bind biotinylated immunoglobulins, or other biotinylated molecules such as, for example, peptides/proteins or lectins. Magnetic beads are generally uniform micro- or nanoparticles of Fe₃O₄. These particles are superparamagnetic, meaning that they are attracted to a magnetic field but retain no residual magnetism after the field is removed. Suspended superparamagnetic particles tagged to a cell of interest can be removed from a matrix using a magnetic field, but they do not agglomerate (i.e., they stay suspended) after removal of the field.

A common format for separations involving superparamagnetic nanoparticles is to disperse the beads within the pores of larger microparticles. These microparticles are coated with a monoclonal antibody for a cell-surface antigen. The antibody-tagged, superparamagnetic microparticles are then introduced into a cellular suspension. The particles bind to cells expressing the surface antigen of interest and maybe separated out with the application of a magnetic field. This may be facilitated by running the suspension over a high gradient magnetic separation column placed in a strong magnetic field. The magnetically labeled cells are retained in the column while non-labeled cells pass through. When the column is removed from the magnetic field, the magnetically retained cells are eluted. Both, labeled and non-labeled fractions can be completely recovered.

iii. Affinity Chromatography

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

The matrix should be a substance that does not adsorb molecules to any significant extent and that has a broad range of chemical, physical and thetinal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed elsewhere in this document.

C. Embryonic Stem Cell Lines

Embryonic stem cell lines (ESC lines) are cultures of cells derived from the epiblast tissue of the inner cell mass (ICM) of a blastocyst or earlier morula stage embryos. A blastocyst is an early stage embryo—approximately four to five days old in humans and consisting of 50-150 cells. ESCs are pluripotent and give rise during development to all derivatives of the three primary germ layers: ectoderm, endoderm and mesoderm. In other words, they can develop into each of the more than 200 cell types of the adult body when given sufficient and necessary stimulation for a specific cell type. They do not contribute to the extra-embryonic membranes or the placenta.

Typically, stem cells have been maintained using tissue culture methods that essentially date from the 1950's. In particular, they are often fed using mouse embryonic fibroblasts (“feeder cells”) while being simultaneously suspended in a nutrient solution. However, many scientists are recognizing the importance of using media that is completely free of animal ingredients. This not only liberates cell lines from animal feeder cells, but also brings the in vivo therapeutic use of stem cells one step closer to reality.

As of March 2007, there were 21 independent, fully-developed stem cell lines available for widespread distribution to researchers. The sources include BresaGen, Inc. (hESBGN-01, hESBGN-02, hESBGN-03), Cellartis AB (Sahlgrenska 1, Sahlgrenska 2), ES Cell International (HES-1, HES-2, HES-3, HES-4, HES-5, HES-6), Technion-Israel Institute of Technology (I 3, I 4, I 6), University of California, San Francisco (HSF-1, HSF-6), and Wisconsin Alumni Research Foundation/WiCell Research Institute (H1, H7, H9, H13, H14).

### II. CULTURE COMPONENTS

A. Macrolide Antibiotics

The macrolides are a group of antibiotics whose activity stems from the presence of a macrolide ring, a large macrocyclic lactone ring to which one or more deoxy sugars, usually cladinose and desosamine, may be attached. The lactone rings are usually 14, 15 or 16-membered. The mechanism of action of the macrolides is inhibition of bacterial protein biosynthesis by binding reversibly to the subunit 50S of the bacterial ribosome, thereby inhibiting translocation of peptidyl tRNA. This action is mainly bacteriostatic, but can also be bactericidal in high concentrations. Macrolides tend to accumulate within leukocytes, and are therefore actually transported into the site of infection.

Common antibiotic macrolides include azithromycin (Zithromax, Zitromax, Sumamed), clarithromycin (Biaxin), dirithromycin (Dynabac), erythromycin, and roxithromycin (Rulid, Surlid, Roxid). Developmental macrolides include carbomycin A, josamycin, kitasamycin, midecamicine/midecamicine acetate, oleandomycin, spiramycin, troleandomycin, and tylosin/tylocine (Tylan). Ketolides are a new class of antibiotics that are structurally related to the macrolides. They include telithromycin (Ketek), and cethromycin. Non-antibiotic macrolides include tacrolimus (Prograf), which is used as an immunosuppressant, is also a macrolide. It has similar activity to cyclosporin.

B. Rho-Associated Kinases and Inhibitors Thereof

Rho-associated kinases are serine/threonine-specific protein kinases which are activated by GTP-bound RhoA. Common inhibitors include HA-1077 and hydroxyfausudil. Also describing Rho Kinase inhibitors are U.S. Pat. Nos. 6,943,172, 6,924,290, and 7,041,687, and U.S. Patent Publications 2007/0270386, 2007/0135466, 2006/0122185, 2005/0234059, 2005/0182061, 2004/0029857, and 2004/0009968, 2007/0238741, 2006/0142314, 2006/0142313, 2006/0105042, 2006/0084591, 2005/0209261, 2005/0192304, 2004/0121011, 2004/0014755, 2004/0002507, 2004/0002508, 2003/0220357, 2003/0125344 and 2003/0087919, each of which are hereby incorporated by reference.

C. Dissociation Treatments

In order to reduce aggregation, the hESCs or iPSCs may be treated with dissociation agents such as collagenase, trypsin, dispase, versene, or TrypLE™, or Accutase.

Collagenases are enzymes that break the peptide bonds in collagen. They assist in destroying extracellular structures in pathogenesis of bacteria such as Clostridium spp. They are an exotoxin (a virulence factor) and help to facilitate the spread of gas gangrene. They normally target the connective tissue in muscle cells and other body organs.

Trypsin is a serine protease found in the digestive system, where it breaks down proteins. Trypsin predominantly cleaves peptide chains at the carboxyl side of the amino acids lysine and arginine, except when either is followed by proline. It is used for numerous biotechnological processes. The process is commonly referred to as trypsin proteolysis or trypsinization.

TrypLE™ is a recombinant enzyme replacing animal trypsin for the dissociation of adherent mammalian cells from plastic. TrypLE™ Select works on a wide range of cells and requires minimal protocol changes. Inactivation with trypsin inhibitors is not required. It is provided by Invitrogen as a 1× concentration in a PBS-EDTA buffer and is phenol red-free.

Dispase is an protease which cleaves fibronectin, collagen IV, and to a lesser extent collagen I. It is found in some bacteria and can be isolated from culture filtrates of Bacillus polymyxa. It can be extracted, purified, and used in research. It can be particularly useful to separate embryonic epithelia and mesenchyme. Dispase II is specific for the cleavage of leucine-phenylalanine bonds.

Versene is the trade name for EDTA, and a variety of different forms. VERSENE™ 100 chelating agent is an aqueous solution of the tetrasodium salt of ethylenediaminetetraacetic acid (Dow). Na₄EDTA is the strongest, most versatile, and widely used chelant for controlling metal ions over a broad pH range in aqueous systems.

Accutase™ (Innovative Cell Technologies, San Diego, Calif.) is an enzyme cell detachment medium supplied in Dulbecco's PBS containing 0.5 mM EDTA4Na and phenol red. Accutase™ has both protease and collagenolytic activity. Accutase has been extensively used in research and biopharmaceutical laboratories.

### III. EMBRYONIC STEM CELL EXPANSION

A. Culture

Cell culture facilitates the maintenance, propagation and expansion of cells in vitro under controlled conditions. Cells may be cultured in a variety of types of vessels constructed of, for example, glass, plastic or metal. The surfaces of culture vessels may be pre-treated or coated with, for example, collagen, polylysine, or components of the extracellular matrix, to facilitate the cellular adherence. Some sophisticated techniques utilize entire layers of adherent cells, feeder cells, which are used to support the growth of cells with more demanding growth requirements.

Cells are normally cultured under conditions designed to closely mimic those observed in vivo. In order to mimic the normal physiological environment cells are generally incubated in a CO₂ atmosphere with semi-synthetic growth media. Culture media is buffered and contains, among other things, amino acids, nucleotides, salts, vitamins, and also a supplement of serum such as fetal calf serum (FCS) horse serum or even human serum. Culture media may be further supplemented with growth factors and inhibitors such as hormones, transferrin, insulin, selenium, and attachment factors.

As a rule, cells grown in vitro do not organize themselves into tissues. Instead, cultured cells grow as monolayers (or in some instances as multilayers) on the surface of tissue culture dishes. The cells usually multiply until they come into contact with each other to form a monolayer and stop growing when they come into contact with each other due to contact inhibition.

Anchorage-dependent cells show the phenomenon of adherence, i.e., they grow and multiply only if attached to the inert surface of a culture dish or another suitable support. Such cells cannot normally be grown without a solid support. Many cells do not require this solid surface and show a phenomenon known as Anchorage-independent growth. Accordingly, one variant of growing these cells in culture is the use of Spinner cultures or suspension cultures in which single cells float freely in the medium and are maintained in suspension by constant stirring or agitation. This technique is particularly useful for growing large amounts of cells in batch cultures.

Anchorage-independent cells are usually capable of forming colonies in semisolid media. Some techniques have been developed that can be used also to grow anchorage-dependent cells in spinner cultures. They make use of microscopically small positively-charged dextran beads to which these cells can attach.

The starting material for the establishment of a cell culture may be a pre-existing cell line or tissue from a suitable donor obtained under sterile conditions. The tissues may be minced and treated with proteolytic enzymes such as trypsin, collagenase of dispase to obtain a single cell suspension that can be used to inoculate a culture dish. In some cases dispersion of tissue is also effectively achieved by treatment with buffers containing EDTA. A particular form of initiating a cell culture is the use of tiny pieces of tissues from which cells may grow out in vitro.

Bioreactor cultured hESC aggregates are dissociated every 6 days with Accutase or a similar agent to regulate aggregate size. This aspect of the invention is described, in particular, aspects, in the Examples below.

B. Bioreactors

A bioreactor may refer to any device or system that supports a biologically active environment. More specifically, a bioreactor is defined here as a closed device or system for growth of cells in cell culture. On the basis of mode of operation, a bioreactor may be classified as batch, fed batch or continuous (e.g., continuous stirred-tank reactor model). Cells growing in bioreactors may be suspended or immobilized. The simplest, where cells are immobilized, is a dish, and large scale immobilized cell bioreactors are moving media, packed bed, fibrous bed and membrane.

Bioreactor design is a complex engineering task. Under optimum conditions, the microorganisms or cells are able to perform their desired function with 100% rate of success. The bioreactor's environmental conditions like gas (i.e., air, oxygen, nitrogen, carbon dioxide) flow rates, temperature, pH and dissolved oxygen levels, and agitation speed/circulation rate need to be closely monitored and controlled.

In continuous flow stirred tank reactors (CSTR or chemostat), fresh medium is fed into the bioreactor at a constant rate, and medium mixed with cells leaves the bioreactor at the same rate. A fixed bioreactor volume is maintained and, ideally, the effluent stream should have the same composition as the bioreactor contents. The culture is fed with fresh medium containing one and sometimes two growth-limiting nutrients such as glucose. The concentration of the cells in the bioreactor is controlled by the concentration of the growth-limiting nutrient. A steady state cell concentration is reached where the cell density and substrate concentration are constant. The cell growth rate (g) is controlled by the dilution rate (D) of growth limiting nutrient.

Cell culture bioreactors are categorized into two types: those that are used for cultivation of anchorage-dependent cells (e.g., primary cultures derived from normal tissues and diploid cell lines), and those that are used for the cultivation of suspended mammalian cells (e.g., cell lines derived from cancerous tissues and tumors, transformed diploid cell lines, hybridomas). In some cases, the bioreactor may be modified to grow both anchorage-dependent and suspended cells. Ideally, any cell culture bioreactor must maintain a sterile culture of cells in medium conditions which maximize cell growth and productivity. Also, fouling can harm the overall sterility and efficiency of the bioreactor, especially the heat exchangers (see below). To avoid fouling, the bioreactor must be easily cleanable and must be as smooth as possible.

A heat exchanger may be needed to maintain the bioreactor at a constant temperature. Biological processes can generate heat; therefore, in some cases, bioreactors need refrigeration. They can be refrigerated with an external jacket or, for very large vessels, with internal coils.

In aerobic processes, such a cell culture, optimal oxygen transfer must be considered. Oxygen is poorly soluble in water, and is relatively scarce in air (20.8%). Oxygen transfer is usually helped by agitation, which is also needed to mix nutrients and to keep the fermentation homogeneous. There are, however, limits to the speed of agitation, due both to high power consumption (which is proportional to the cube of the speed of the electric motor) and to the damage to organisms caused by excessive tip speed causing shear stress.

Bioreactors facilitate the development of cell and tissue engineering therapies as they enable the generation of clinically-relevant cell numbers in a highly controlled and reproducible culture system. The transition from the expansion of highly undifferentiated stem cells to the differentiation of these cells into specialized cells requires little to no modifications of the bioreactor culture system. Specifically, differentiation agents can be added directly to the culture media to generate specialized cells. Further, scaffolds and matrices can be easily added to many types of bioreactors, including batch, CSTR and perfusion systems, enabling the generation of transplantable tissue products. Finally, bioreactors are easily adapted to meet GMP level standards, thus providing a viable method to transition cell therapies from the bench to the bed-side. For example, the cells can easily be cultured in bioreactors using animal-free, or xeno-free, medium and other culture solutions and using highly aseptic cell culture techniques in a GMP level facility.

C. Cell Differentiation Factors

In certain aspects of the instant invention, cells are cultured with differentiating agents. They may also be cultured after contact, i.e., after they have been induced to differentiate toward a given or specific phenotype. Cells will be cultured under specified conditions to achieve particular types of differentiation, and provided various factors necessary to facilitate the desired differentiation.

Cell growth and differentiation factors are molecules that stimulate stem cells to proliferate and/or promote differentiation of cell types into functionally mature forms. In some embodiments of the invention, cell growth and differentiation factors may be administered in combination with TLR ligands in order to direct the administered cells to proliferate and differentiate in a specific manner. One of ordinary skill would recognize that the various factors may be administered prior to, concurrently with, or subsequent to the administration of TLR ligands. In addition, administration of the growth and/or differentiation factors may be repeated as needed.

It is envisioned that a growth and/or differentiation factor may constitute a hormone, cytokine, hematapoietin, colony stimulating factor, interleukin, interferon, growth factor, other endocrine factor or combination thereof that act as intercellular mediators. Examples of such intercellular mediators are lymphokines, monokines, growth factors and traditional polypeptide hormones. Included among the growth factors are growth hormones such as human growth hoimone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; prostaglandin, fibroblast growth factor; prolactin; placental lactogen, OB protein; tumor necrosis factors-α and -β.; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte/macrophage-CSF (GM-CSF); and granulocyte-CSF (G-CSF); interleukins (ILs) such as IL-1, IL-1α, IL-2, IL-3, IL-4, IL-5, IL-6, IL-7, IL-8, IL-9, IL-11, IL-12; IL-13, IL-14, IL-15, IL-16, IL-17, IL-18. Also contemplated are CD14 or signal transducers of the MyD88 pathway. As used herein, the term growth and/or differentiation factors include proteins from natural sources or from recombinant cell culture and biologically active equivalents of the native sequence, including synthetic molecules and mimetics.

The CD14 antigen is a high affinity receptor for the complex of lipopolysaccharide (LPS) and LPS-Binding protein (LBP). The CD14 antigen is part of the functional heteromeric LPS receptor complex comprised of CD14, TLR4 and MD-2. CD14 is strongly expressed on most human monocytes and macrophages in peripheral blood, other body fluids and various tissues, such as lymph nodes and spleen. CD14 is weakly expressed on subpopulations of human neutrophils and myeloid dendritic cells.

The MD-2 protein appears to associate with Toll-like receptor 4 on the cell surface and confers responsiveness to lipopolysaccharide (LPS), thus providing a link between the receptor and LPS signaling. Basic amino acid clusters in MD-2 are involved in cellular lipopolysaccharide recognition.

Other factors include Wnt/β-catenin signal-, Notch signal- or Hedgehog signal-promoting agents. Wnt/β-catenin signal-promoting agents include agonists of one or more of Wnt1, Wnt2, Wnt2b/13, Wnt3, Wnt3a, Wnt4, Wnt5a, Wnt5b, Wnt6, Wnt7a, Wnt7b, Wnt7c, Wnt8, Wnt8a, Wnt8b, Wnt8c, Wnt10a, Wnt10b, Wnt11, Wnt14, Wnt15, or Wnt16. Notch signal-promoting agents include agonists of Notch, Delta, Serrate, Jagged, Deltex, Mastermind, Enhancer of Split, Hes1, Split, Hairless, Suppressor of Hairless, or RBP-Jk. The Wnt/β-catenin signal-, Notch signal- or Hedgehog signal-promoting agent may a glycogen synthase kinase (GSK) inhibitor.

Other factors include chemical agents such as retinoic acid and lithium chloride.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials & Methods

Maintenance culture of hESCs. The H9 hESC line was used in accordance with the Canadian hESCs research guidelines. Undifferentiated hESCs were maintained as described previously. Cells were cultured in static tissue culture flasks on a feeder layer of mouse embryonic fibroblast (MEF) cells inactivated with 10 μg/ml Mitomycin C (Sigma), or coated with Matrigel™ in 35-mm gelatin-coated tissue culture dishes (Nunc), with mTeSR (Stemcell Technologies) under standard conditions (37° C., 5% CO₂, saturated humidity). The hESCs were passaged as small clumps by enzymatic dissociation and subcultured on fresh MEF feeder layers every 6 days. In brief, the colonies were treated with 0.1 mg/ml collagenase IV (Invitrogen) in mTeSR at 37° C. for 30 min followed by TrypLE Select (Invitrogen) at room temperature for 2 min, gently rinsed with 5 ml Dulbecco's phosphate-buffered saline (DPBS; Invitrogen), followed by adding 2 ml culture medium and gently pipetting them several times, and then broken into small clumps and passaged at a 1:3 ratio.

Bioreactor Maintenance of hESCs. H9 cells passaged in static on Matrigel™ or using feeder layers were pre-treated for 1 hr with 10 μM Y-27632 (Sigma), then dissociated as described earlier. The hESCs were then inoculated into 125 ml bioreactors (NDS Technologies) at 1.8×10⁴ cells/ml. The bioreactors contained 100 ml of mTeSR medium supplemented with 10 μM Y-27632 and 0.1 nM Rapamycin (Sigma), were agitated at 100 rpm and were incubated under standard conditions (37° C., 5% CO₂, saturated humidity). After 24 hrs in suspension culture, the Y-27632 was removed and replaced with mTeSR medium containing 0.1 nM Rapamycin. Every 6 days the hESC aggregates were passaged by removing the entire contents of the bioreactor and dissociating the cells using Accutase (Millipore) in the presence of 10 μM Y-27632 until a single cell suspension was achieved. The single cell suspension of hESCs was split 1:5 and inoculated into a new bioreactor containing 100 ml of mTeSR medium supplemented with 10 μM Y-27632 and 0.1 nM Rapamycin for 24 hrs.

Karyotype analysis. Karyotype analyses of hESCs were carried out using G-banding method. In brief, cells were incubated with 0.2 μg/ml colcemid at 37° C. for 30 min, trypsinized, resuspended, and incubated in 0.068 M KCl for 25 min at 37° C., then 2 rinsed with 3:1 methanol:glacial acetic acid three times and dropped to make the spread of chromosomes on the slides. The dried slides were baked overnight at 55° C., treated with 0.05% trypsin for 30 s to 2 min, and stained with Giemsa and Leishman's solution.

Immunofluorescence. Aliquots of bioreactor generated aggregates were washed in PBS and fixed in 4% PFA in PBS at 4° C. overnight. Aggregates where then permeabilized in 0.5% saponin in PBS at 4° C. overnight, rinsed once in PBS, then blocked in 3% BSA again at 4° C. overnight. Primary antibodies (Oct-4, Nanog, Tra-1-60, Tra-1-81, SSEA-4: all Santa Cruz) were diluted 1:50 in 3% BSA, added to the cell samples and incubated overnight at 4° C. The aggregates were then washed 3 times with PBS and blocked again overnight at 4° C. Following the block, the aggregates were incubated with an appropriate alexa-fluor 488 secondary antibody (Molecular Probes) and Toto-3 (Molecular Probes) overnight at 4° C. After incubation, the aggregates were washed 3 time with PBS and mounted on slides with mountant (9:1 glycerol:PBS). Spacers (270 μm) were adhered to slides prior to mounting to avoid aggregate compression. Slides were analyzed using a Zeiss 510 confocal Microscope with 488, 568 and 633 nm filters. Images where prepared using Zeiss LSM image browsing software.

Teratoma formation. Fox Chase CB-17 SCID mice were obtained from Charles River and housed in the single-barrier animal facility of the Faculty of Medicine, University of Calgary. Mice were fed ad libitum with a standard diet and water. One million cells were injected into the skin fold of the inner thigh of six mice per group. The animals were sacrificed and emerging tissue material was dissected. Animal protocols were carried out as approved by the University of Calgary animal protocol #M03038. Excised tissues were fixed overnight in 4% PFA at 4° C. and then embedded in paraffin. Sections were stained in hematoxylin/eosin according to standard procedures.

Flow Cytometry. hESCs were dissociated into a single cell suspension and subjected to fluorescence-activated cell sorting using a FACS Calibur instrument and the CellQuest software from Becton Dickinson (Germany). Ten thousand events were registered per sample and analysis of whole cells was performed using appropriate scatter gates to avoid cellular debris and aggregates. Cell were stained using the following antibodies: Oct4, Nanog, Tra-1-60, Tra-1-81, SSEA-4 (all Santa Cruz). All primary antibodies were directly conjugated to R-Phycoerythrin (R-PE) or Alexa Fluor 488 using Zenon conjugation kits (Invitrogen) following the manufactures method. Anti-mouse or anti-goat IgG R-PE or FITC were used as isotope controls depending on the primary conjugation molecule used.

In Vitro Differentiation. Cells were replated on MEF feeder layers. The resulting multilayer colonies were cut into small clumps using a stem cell passaging tool (Invitrogen), and cultured in 35 mm agar-coated dishes (Nunc). The differentiation medium consisted of 80% Dulbecco's Modified Eagle's Medium (DMEM, Invitrogen), 20% fetal bovine serum (FBS, Invitrogen), 1 mM L-Glutamine, 0.1 mM (3-mercaptoethanol, and 0.1 mM non-essential amino acids. After 7-14 days, the cells aggregated and generated cystic embryoid bodies (EBs). RT-PCR analysis on undifferentiated cells and day 16 EBs were performed by using primers specific for the three germ layers and pluripotency as previously described, namely, Nanog, Rex1, Oct-4, and Sox2, ectoderm (neurofilament-68 and keratin), mesoderm enolase and kallikrein) and endoderm (α-fetoprotein and α1-antitrypsin).

### Example 2

### Results

Human embryonic stem cells (hESCs) are potentially one of the most useful cell types for cell and tissue engineering therapies as a result of their pluripotency and their ability to self-renew. The inventors, along with other research groups, have previously developed effective bioreactor protocols for the large-scale expansion of highly pluripotent murine ESCs (Cormier et al., 2006; zur Nieden et al., 2007; Fok et al., 2006; Abranches et al., 2007). One study, by Cameron et al. (2006), used stirred suspension cultures to expand preformed embryoid bodies from hESCs. However, to date, there have been no reported studies on the development of bioreactor expansion protocols for pluripotent hESCs. There are several limitations that have prevented the development of these techniques for hESCs. For example, the need to provide cells with an adequate oxygen supply must be balanced against the detrimental effects of hydrodynamic shear stress that is created in the stirred environment. Also, hESCs are particularly sensitive to dissociation, which is required for passaging, cryopreservation, and other applications, both in static and in bioreactor culture systems.

Recently, it has been discovered that an inhibitor of Rho Kinase (ROCK inhibitor; Y-27632) increases the survival rate of dissociated, single hESCs. This breakthrough has allowed for the development of new methods in hESC culture, with the promise of increasing hESC numbers into the realm of clinical relevance. Using ROCK inhibitor for 24 hrs and continuous treatment with 0.1 nM Rapamycin, the inventors have been able to transition hESCs from static culture systems into stirred suspension bioreactors and maintained cultures with high expression levels of Nanog, Oct-4 and other markers of pluripotency, a normal karyotype and the ability to form teratomas in vivo.

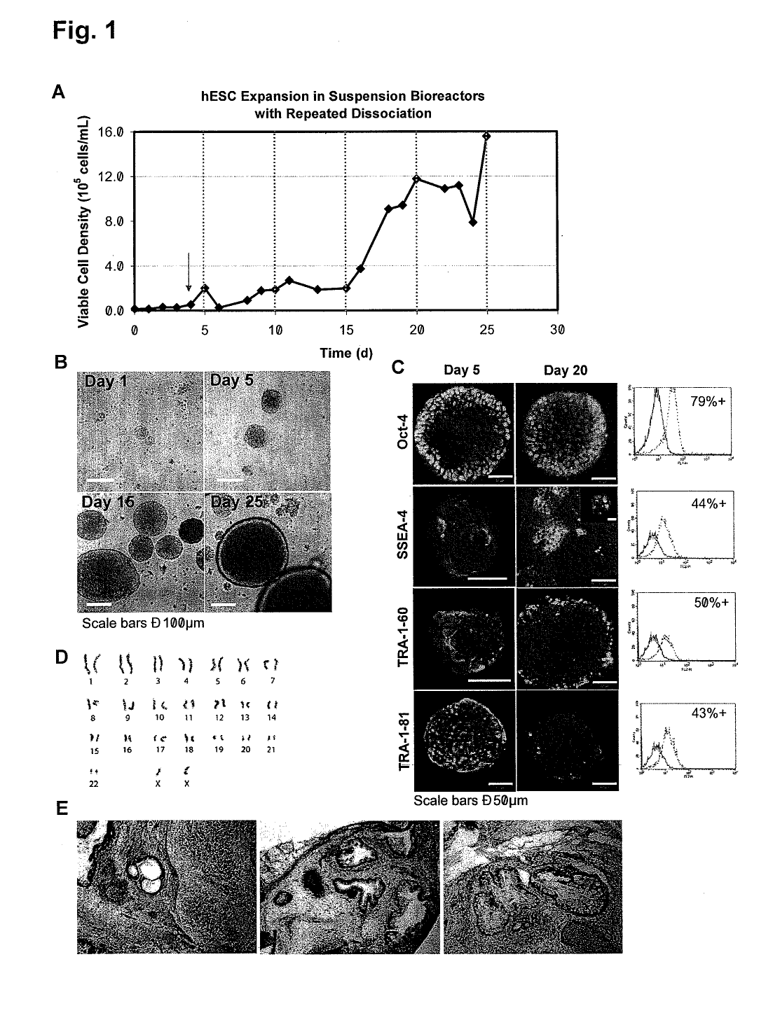

In the bioreactor systems, hESCs (collected from static cultures containing Matrigel™ or human foreskin fibroblast feeder (HFF) cells) were inoculated at 1.8×10⁴ cells/ml into 100 ml of mTeSR medium (Stemcell Technologies Inc.) and agitated at 100 rpm. Under these conditions, and in the presence of 10 μM ROCK inhibitor and 0.1 nM Rapamycin, the hESCs form tight aggregates, similar to what the inventors had observed with the mouse ESCs (FIG. 1B) (Cormier et al., 2006). When ROCK inhibitor was excluded from the bioreactor cultures, noticeably fewer aggregates were observed and mainly single cells were observed by day 5 (data not shown). Moreover, when the concentration of Rapamycin was reduced to 0.05 nM hESC aggregates became irregular and no viable cells were present after the first passage (day 6-7) (data not shown).

Initially, the hESC bioreactor cultures were treated with ROCK inhibitor from day 0 to day 4 and the aggregates were dissociated into smaller clumps every 5 days using collagenase. The overall expansion in these non-passaged cultures was 67-fold over 20 days (FIG. 1A). Although this method lead to effective hESC expansion, collagenase treatment was not sufficient for the dissociation of the hESC aggregates into small enough clumps. This resulted in aggregates that were overgrown and cultures with decreased expression of pluripotency markers (Oct-4, SSEA-4, TRA-1-60 and TRA-1-81) over the expansion period (FIGS. 1B-C). FACS analysis demonstrated that approximately 50% of the cells expressed SSEA-4, TRA-1-60 and TRA-1-81 by day 20 and 79% of the cells were positive for Oct-4 (FIG. 1C). Importantly, cytoplasmic Oct-4 expression (i.e., non-transcriptional function) was observed in some aggregates, which may exaggerate the FACS results. Although there was a substantial drop in the number of pluripotent cells in the bioreactors by day 20, these cultures retained a normal karyotype and the ability to form teratomas in vivo (FIGS. 1D-E).

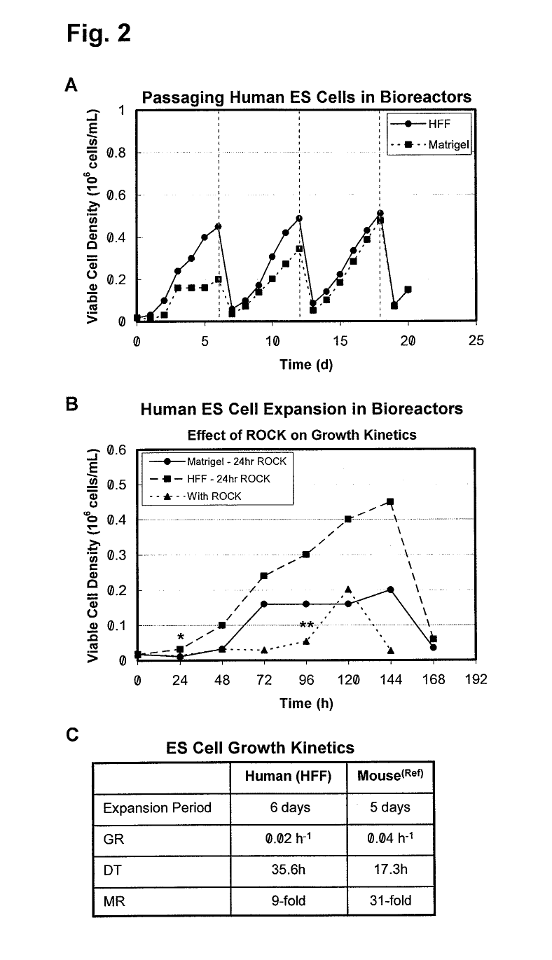

To achieve high levels of pluripotency in the hESC bioreactor cultures, smaller aggregate diameters were maintained by passaging the cultures every 6 days. Here, the cultures were dissociated into single cells, using Accutase (Millipore), and were then split 1:5. For each passage, the cells were treated with ROCK inhibitor for the first 24 hours only, following their dissociation into single cells (FIG. 2A). This method was used to expand hESCs that had been collected from static culture systems containing either Matrigel™ or human foreskin fibroblast feeder cells (HFFs) (Meng et al., 2008). The hESCs collected from static HFF cultures possessed reproducible growth characteristics over each passage in the bioreactors. Interestingly, the hESCs collected from static Matrigel™ cultures did not expand well over the first passage but over subsequent passages acquired a growth curve similar to that of the hESCs from the HFF cultures (FIG. 2A). By decreasing the exposure to ROCK inhibitor from 4 days to 24 hrs, a substantial decrease in the lag phase of the cultures was achieved (FIG. 2B).

With continuous passaging, the hESCs (from HFF cultures) possessed an average growth rate of 0.02 h⁻¹ and expanded 9.4-fold over a 6 day passage period in the bioreactors. In contrast, Cormier et al. (2006) showed that mESCs possess a growth rate of 0.04 h⁻¹ and expanded 31-fold over 5 days (FIG. 2C). The slower growth rate of the hESCs compared to the mouse ESCs in the bioreactor cultures mimics the difference in growth kinetics observed in static culture systems.

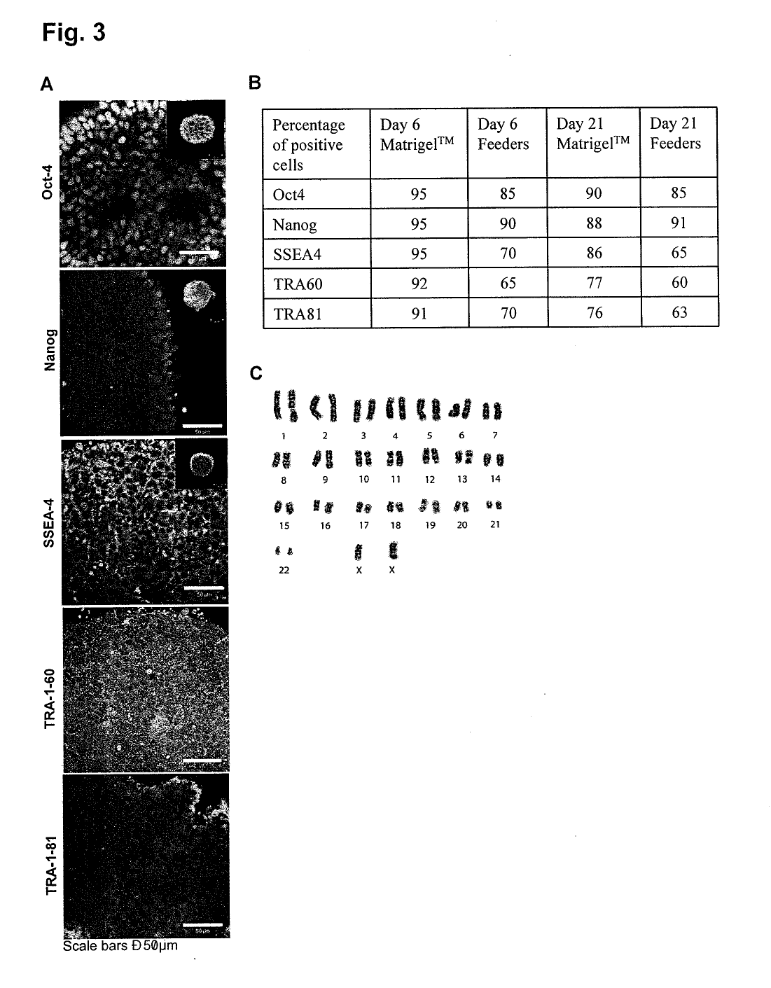

Although there were differences observed in the growth kinetics of the Matrigel™ versus the HFF cultured hESCs over the first passage in the bioreactors, the inventors found no appreciable difference in the expression of all pluripotency markers on day 6. To visualize the localization and expression pattern of the pluripotency markers within aggregates by day 21 of expansion in the bioreactors we performed whole mount immunofluorescence using confocal microscopy. The inventors found that under regular passaging conditions (every 6 days) the inventors obtained uniform expression of all pluripotency markers tested within aggregates by day 21 of bioreactor culture (FIG. 3A). FACS analysis of the day 6 and day 21 bioreactor cultures showed higher positive expression levels of the pluripotency markers compared to the non-passaged cultures, with the many of the markers being expressed at levels greater than 90%, however, hESC aggregates generated from Matrigel™ conditions contained greater numbers of pluripotent cells on days 6 and 21 (FIG. 3B). Furthermore, there were no detectable differences between expression and localization of markers between aggregates generated from hES cells cultured on Matrigel™ or HFFs. Correspondingly, both groups maintained a normal karyotype and produced teratomas in vivo (FIGS. 3C-D).

A signficant goal of any cell culture method that can potentially produce therapeutic grade cells is the elimination of animal serum. The present inventor tested the following culture media that lacks any animal serum:

TABLE 2
ANIMAL SERUM FREE MEDIUM

| | | |
|---|---|---|
| TF1 medium | 100 | ml |
| 80 mls DMEM/F12 | 80 | ml |
| Human serum albumin | 1.3 | g |
| Thiamine | 0.67 | mg |
| Glutathione | 0.2 | mg |
| Insulin | 6.6 | mg |
| Transferrin | 1.1 | mg |
| bFGF | 10 | μg |
| TGF-β | 0.6 | μg |
| Pipecolic acid | 10 | μg |
| glutamax | 14.6 | mg |
| β-mercaptoethanol | 0.7 | μl |
| Non-essential Amino Acids | 1 | ml |
| NaHCO3 | 56 | mg |
| GABA | 10 | mg |
| LiCl | 4.25 | mg |
| Chemically Defined Lipid | 1 | ml |
| Concentrate | | |
| Pen/Strep | 1 | ml |
| 1M NaOH | 750 | μl |

All of the compositions and/or methods disclosed and claimed herein can be made and executed without undue experimentation in light of the present disclosure. While the compositions and methods of this invention have been described in temis of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### V. REFERENCES

The following references, to the extent that they provide exemplary procedural or other details supplementary to those set forth herein, are specifically incorporated herein by reference.
U.S. Pat. No. 3,826,364
U.S. Pat. No. 4,284,412
U.S. Pat. No. 4,498,766
U.S. Pat. No. 4,661,913
U.S. Pat. No. 4,714,682
U.S. Pat. No. 4,767,206
U.S. Pat. No. 4,774,189
U.S. Pat. No. 4,857,451
U.S. Pat. No. 4,989,977
U.S. Pat. No. 5,160,974
U.S. Pat. No. 5,478,722
U.S. Pat. No. 6,924,290
U.S. Pat. No. 6,943,172
U.S. Pat. No. 7,041,687
U.S. Patent Publn. 2003/0087919
U.S. Patent Publn. 2003/0125344
U.S. Patent Publn. 2003/0220357
U.S. Patent Publn. 2004/0002507
U.S. Patent Publn. 2004/0002508
U.S. Patent Publn. 2004/0009968
U.S. Patent Publn. 2004/0014755
U.S. Patent Publn. 2004/0029857
U.S. Patent Pubin. 2004/0121011
U.S. Patent Pubin. 2005/0182061
U.S. Patent Publn. 2005/0192304
U.S. Patent Publn. 2005/0209261
U.S. Patent Publn. 2005/0234059
U.S. Patent Publn. 2006/0084591
U.S. Patent Publn. 2006/0105042
U.S. Patent Pubin. 2006/0122185
U.S. Patent Pubin. 2006/0142313
U.S. Patent Pubin. 2006/0142314
U.S. Patent Pubin. 2007/0135466
U.S. Patent Pubin. 2007/0238741
U.S. Patent Pubin. 2007/0270386
Abranches et al., Biotechnol. Bioeng., 96:1211-1221, 2007.
Asahara et al. EMBO J., 18(14):3964-3972, 1999.
Asahara et al., Science, 275:964-967, 1997.
Cameron et al., Biotechnol. Bioeng., 94:938-948, 2006.
Cormier et al., Tissue Eng., 12:3233-3245, 2006.
Fok and Zandstra, Stem Cells, 23:1333-1342, 2005.
Hristov et al., Arterioscler Thromb. Vasc. Biol., 23(7):1185-1189, 2003.
King and Miller, Curr. Opin. Chem. Biol., 11:1-15, 2007.
Meng et al., Mol. Reprod. Dev., 75(4):614-622, 2008.
PCT Appln. WO/2005/078073
Shaw et al., Blood Cells Mol. Dis., 32(1):168-175, 2004.
Thomson et al., Science, 282:1145-1147, 1998.
Werner et al., N. Engl. J. Med., 353:999-1007, 2005.
zur Nieden et al., J. Biotechnol., 129(3):421-432, 2007.

1. A method for expanding stem cells in culture comprising:
(a) providing a starting culture comprising human embryonic stem cells (hESCs) or induced pluripotent stem cells (iPSCs);
(b) incubating said starting culture with a macrolide antibiotic and a Rho and Rho family kinase inhibitor for a period of time and under conditions suitable to permit expansion of said hESCs or iPSCs.

2. The method of claim 1, wherein said macrolide antibiotic is selected from the group consisting of sirolimus, tacrolimus, cyclosporine, everolimus, ascomycin, erythromycin, azithromycin, clarithromycin, clindamycin, lincomycin, dirithromycin, josamycin, spiramycin, diacetyl-midecamycin, tylosin, roxithromycin, ABT-773, telithromycin, leucomycins and lincosamide.

3. The method of claim 1, wherein said starting culture is comprised in a bioreactor.

4. The method of claim 1, wherein said starting culture is comprised in a static vessel.

5. The method of claim 3, wherein said bioreactor is a micro-bioreactor.

6. The method of claim 3, wherein said bioreactor is a commercial bioreactor.

7. The method of claim 3, wherein said bioreactor has a volume of between 100 μl and 1000 L.

8. The method of claim 1, wherein step (a) comprises preparing said starting culture by inoculating said bioreactor with hESCs or iPSCs from a static culture.

9. The method of claim 1, wherein said macrolide antibiotic in present at about 0.1 nM to 100 nM.

10. The method of claim 1, wherein said period of time comprises 2, 3, 4, 5, 6, 7, 8, 9, 10, 11, 12, 13, 14, 15, 16, 17, 18, 19, 20, 21, 22, 23, 24, 25, 30, 40 or 50 days.

11. The method of claim 1, wherein incubating comprises periodic treatment with a dissociation agent to reduce aggregate size.

12. The method of claim 11, wherein said dissociation agent is collagenase, trypsin, dispase, EDTA, or TrypLE™, or Accutase™.

13. The method of claim 1, wherein the Rho and Rho family kinase inhibitor is a ROCK inhibitor or C-3 toxin.

14. The method of claim 1, wherein and Rho and Rho family kinase inhibitor is removed at about 1 day following initiation of incubation.

15. The method of claim 1, wherein said starting culture comprises about 10⁴ to 10⁷ cells.

16. The method of claim 1, wherein expansion comprises 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 60-fold, or 70-fold expansion.

17. The method of claim 1, wherein expansion comprises 80-fold, 90-fold, 100-fold, 125-fold, 150-fold, 200-fold, 400-fold, 500-fold or 1000-fold expansion.

18. The method of claim 1, further comprising inducing said hESCs or iPSCs to differentiate.

19. The method of claim 18, wherein inducing comprises differentiation into immune cells.

20. The method of claim 18, wherein inducing comprises differentiation into neuronal cells.

21. The method of claim 18, wherein inducing comprises differentiation into cardiovascular cells.

22. The method of claim 18, wherein inducing comprises differentiation into muscular cells, skeletal cells, islet cells, bone cells, or cartilage cells.

23. The method of claim 1, wherein said starting culture comprises mTeSR medium.

24. The method of claim 1, wherein said starting culture comprises animal-free medium or xeno-free medium.

25. A method for expanding stem cells in culture comprising:
(a) providing a bioreactor comprising a culture media, human embryonic stem cells (hESCs) or induced pluripotent stem cells (iPSCs), and a macrolide antibiotic;
(b) incubating said bioreactor for a period of time and under conditions suitable to permit expansion of said ESCs or iPSCs by about 10- to 1000-fold.
